(19) United States
(12) Patent Application Publication
(10) Pub. No.: US 2019/0159753 A1
(43) Pub. Date: May 30, 2019

(54) Motor-Assisted Needle Guide Assembly for Ultrasound Needle Placement

(71) Applicant: Avent, Inc., Alpharetta, GA (US)

(72) Inventors: Kenneth C. Hsu, Tustin, CA (US); Shane A. Duffy, Irvine, CA (US)

(21) Appl. No.: 16/321,154

(22) PCT Filed: Aug. 2, 2017
(86) PCT No.: PCT/US2017/045011

§ 371 Date: Jan. 28, 2019

Related U.S. Application Data
(60) Provisional application No. 62/369,823, filed on Aug. 2, 2016.

(51) Int. Cl. A61B 8/08; A61B 17/34

(52) U.S. Cl. CPC A61B 8/0841; A61M 5/46; A61B 2017/3413; A61B 17/3403

(57) ABSTRACT

The present invention is directed to an ultrasound imaging system having a motor-assisted needle guide assembly for easier needle placement during an ultrasound-guided medical procedure. The ultrasound imaging system includes an ultrasound probe having a transducer housing, a transducer transmitter, a needle guide assembly communicatively coupled to the ultrasound probe, at least one actuator component configured with the needle guide assembly, and a controller. Thus, the controller is configured to determine an insertion angle and a lateral position for the needle guide assembly with respect to the ultrasound probe based on the target site and control the actuator component based on the insertion angle and the lateral position so as to locate the needle guide assembly at the target site during a medical procedure.

### RELATED APPLICATIONS

The present application claims priority to U.S. Provisional Application Ser. No. 62/369,823 filed on Aug. 2, 2016, which is incorporated herein in its entirety by reference hereto.

### FIELD OF THE INVENTION

The present invention relates generally to the field of medical imaging, and more particularly, to a motor-assisted needle guide assembly for easier needle placement during an ultrasound-guided medical procedure.

### BACKGROUND

In conventional ultrasound imaging, a focused beam of ultrasound energy is transmitted into body tissues to be examined and the returned echoes are detected and plotted to form an image. Modern ultrasounds may include two-dimensional (2D) as well as three-dimensional (3D) capabilities. More specifically, some modern ultrasound systems have 3D capabilities that scan a pulsed ultrasound beam in two side-wards directions relative to a beam axis. Time of flight conversion gives the image resolution along the beam direction (range), while image resolution transverse to the beam direction is obtained by the side-wards scanning of the focused beam. With such 3D imaging, a user can collect volume ultrasound data from an object and visualize any cross-section of the object through computer processing. This enables selection of the best 2D image planes for a diagnosis.

Previously, ultrasound imaging was not particularly helpful during nerve block procedures, as the technology was not efficient as delivering clear images of the nerve block anatomy, surrounding structures, and/or the needle location. However, recent advancements in the field of ultrasound imaging have provided for effective nerve block procedures to be performed using such imaging.

As such, improvements to the field of ultrasound imaging that can further benefit certain medical procedures, such as nerve block procedures, that were previously difficult to view under ultrasound imaging would be advantageous. More specifically, a motor-assisted needle guide assembly for easier needle placement during an ultrasound-guided medical procedure would be welcomed in the art.

### SUMMARY OF THE INVENTION

Objects and advantages of the invention will be set forth in part in the following description, or may be obvious from the description, or may be learned through practice of the invention.

In one aspect, the present invention is directed to an ultrasound imaging system. The ultrasound imaging system includes an ultrasound probe having a transducer housing and a transducer transmitter. The transducer housing includes a body extending from a proximal end to a distal end along a longitudinal axis. Further, the distal end of the body includes an internal cavity. The transducer transmitter is configured within the internal cavity and is configured to scan a target site of a patient. The ultrasound imaging system also includes a needle guide assembly communicatively coupled to the ultrasound probe, at least one actuator component configured with the needle guide assembly, and a controller. Thus, the controller is configured to determine an insertion angle and a lateral position for the needle guide assembly with respect to the ultrasound probe based on the target site and control the actuator component based on the insertion angle and the lateral position so as to locate the needle guide assembly at the target site during a medical procedure.

In one embodiment, the ultrasound imaging system may also include a needle and a catheter. In another embodiment, the actuator component may include a first actuator device configured to move the needle guide assembly with respect to a first axis of the ultrasound probe and a second actuator device configured to move the needle guide assembly with respect to a second axis of the ultrasound probe.

In such embodiments, the controller is further configured to control the first and second actuator devices so as to maintain the needle guide assembly in-plane with respect to the first and second axes of the ultrasound probe. More specifically, in certain embodiments, the first and second actuator devices may include a first motor configured to control the insertion angle and a second motor configured to control the lateral position, respectively.

In further embodiments, the ultrasound imaging system may also include a third actuator device for manipulating the needle guide assembly with respect to a third axis of the ultrasound probe.

In additional embodiments, the controller is further configured to adjust at least one of the insertion angle or the lateral position as a function of the target site as the needle guide assembly is being inserted into the patient during the medical procedure. Further, in particular embodiments, the controller is further configured to determine the insertion angle as a function of a depth of the target site within the patient.

In yet another embodiment, the ultrasound probe is further configured to track the needle guide assembly as the assembly is being inserted into the patient. In further embodiments, the ultrasound imaging system may include a user interface configured to display the target site to a user during the medical procedure. In addition, in certain embodiments, the medical procedure may include a peripheral nerve block procedure.

In another aspect, the present invention is directed to a method for locating a needle guide assembly of an ultrasound imaging system at a target site of a patient during an ultrasound-guided medical procedure. The ultrasound imaging system includes an ultrasound probe, an actuator component configured with the needle guide assembly, and a controller. Thus, the method includes placing the ultrasound probe on a patient's skin. Further, the method includes determining, via the controller, an insertion angle and a lateral position for the needle guide assembly with respect to the ultrasound probe based on the target site. The method also includes inserting the needle guide assembly into the patient at the insertion angle. In addition, the method includes controlling, via the controller, the actuator component as the needle guide assembly is being inserted into the patient so as to manipulate the insertion angle and the lateral position of the needle guide assembly. Moreover, the method includes locating, via the controller, the needle guide assembly at the target site.

In one embodiment, the method may include scanning, via the ultrasound probe, images of the target site and generating, via the controller, an ultrasound image based on the images during the ultrasound-guided medical procedure.

In another embodiment, the method may include controlling, via the controller, first and second actuator devices of the actuator component so as to maintain at least one of the needle guide assembly in-plane with respect to the first and second axes of the ultrasound probe. More specifically, in certain embodiments, the first and second actuator devices may include a first motor configured to control the insertion angle and a second motor configured to control the lateral position, respectively. In addition, in particular embodiments, the method may further include manipulating, via a third actuator device, the needle guide assembly with respect to a third axis of the ultrasound probe.

In additional embodiments, the method may also include adjusting, via the controller, at least one of the insertion angle or the lateral position as a function of the target site as the needle guide assembly is being inserted into the patient during the medical procedure.

In yet another embodiment, the method may include determining, via the controller, the insertion angle as a function of a depth of the target site within the patient. In still a further embodiment, the method may include tracking, via the ultrasound probe, the needle guide assembly as the assembly is being inserted into the patient.

In another embodiment, the method may also include generating, via a user interface, a display of the target site for a user. Further, in certain embodiments, the ultrasound-guided medical procedure comprises a peripheral nerve block procedure.

In another aspect, the present invention is directed to a method for controlling a needle guide assembly of an ultrasound imaging system during an ultrasound-guided medical procedure. The ultrasound imaging system includes an ultrasound probe, an actuator component configured with the needle guide assembly, and a controller. Thus, the method includes determining, via the controller, an insertion angle and a lateral position for a needle guide assembly with respect to the ultrasound probe based on the target site. Further, the method includes controlling, via the controller, the actuator component as the needle guide assembly is being inserted into a patient so as to manipulate the insertion angle and the lateral position of the needle guide assembly. It should be understood that the method may further include any of the additional method steps/or features as described herein.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a schematic diagram of one embodiment of an ultrasound imaging system according to the present disclosure;

FIG. 2 illustrates a block diagram of one embodiment of suitable components that may be included in a controller of an ultrasound imaging system according to the present disclosure;

FIG. 3 illustrates a schematic diagram of one embodiment of an ultrasound imaging system according to the present disclosure, particularly illustrating an actuator component manipulating a needle guide assembly towards a target site of a patient;

FIG. 4 illustrates a schematic diagram of one embodiment of an ultrasound imaging system according to the present disclosure, particularly illustrating an actuator component manipulating a needle guide assembly towards a target site of a patient; and

FIG. 5 illustrates a flow diagram of one embodiment of a method for locating a needle guide assembly of an ultrasound imaging system at a target site of a patient during an ultrasound-guided medical procedure according to the present disclosure.

### DETAILED DESCRIPTION OF THE INVENTION

Reference will now be made in detail to one or more embodiments of the invention, examples of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

Generally, the present disclosure is directed to an ultrasound imaging system having a motor-assisted needle guide assembly for easier needle placement during an ultrasound-guided medical procedure. The ultrasound imaging system includes an ultrasound probe having a transducer housing, a transducer transmitter, a needle guide assembly communicatively coupled to the ultrasound probe, at least one actuator component configured with the needle guide assembly, and a controller. Thus, the controller is configured to determine an insertion angle and a lateral position for the needle guide assembly with respect to the ultrasound probe based on the target site and control the actuator component based on the insertion angle and the lateral position so as to locate the needle guide assembly at the target site during a medical procedure.

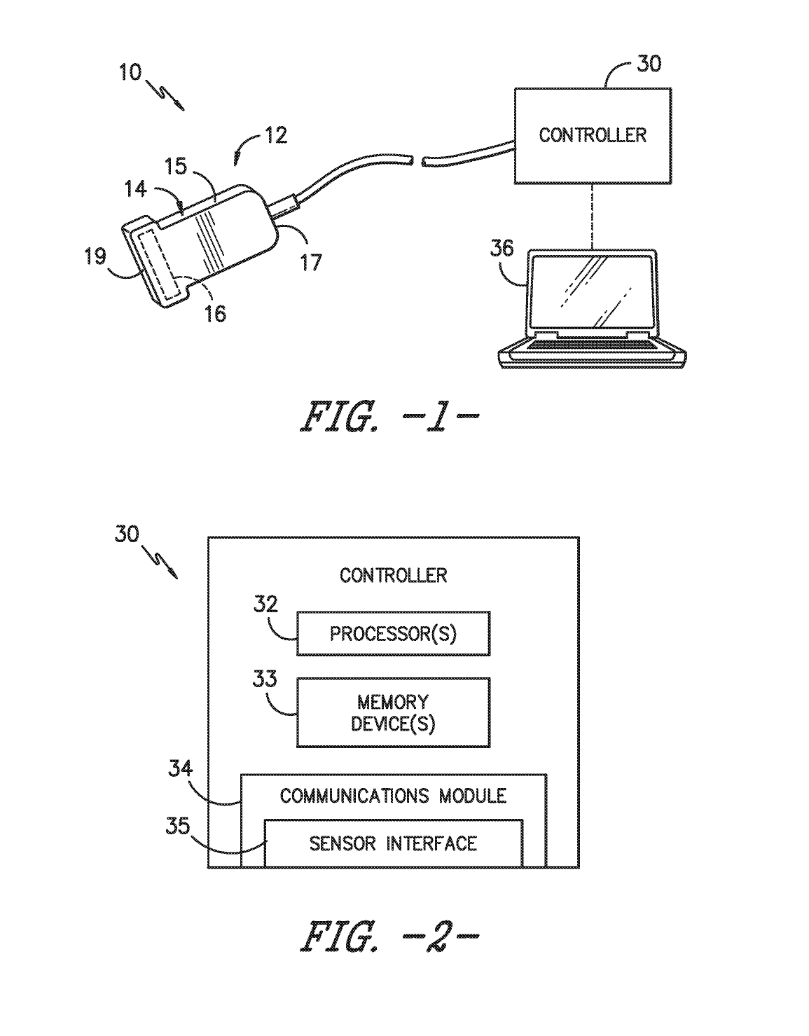

Referring now to the drawings, FIG. 1 illustrates a schematic diagram of one embodiment of an ultrasound imaging system 10 for use during a medical procedure according to the present disclosure. For example, in certain embodiments, the medical procedure may include a peripheral nerve block procedure. Further, as shown, the ultrasound imaging system 10 includes an ultrasound probe 12. More specifically, as shown in FIGS. 1, 3, and 4, the ultrasound probe 12 has a transducer housing 14 and a transducer transmitter 16 mounted therein. As is generally understood, the transducer transmitter 16 is configured to emit and/or receive ultrasound beams. For example, as shown, the transducer housing 14 includes a body 15 extending from a proximal end 17 to a distal end 19 along a longitudinal axis 21. Further, the distal end 19 of the body 15 includes an internal cavity (not numbered). As such, the transducer transmitter 16 may be configured within the internal cavity such that the transmitter 16 is configured to scan a target site 38 of a patient.

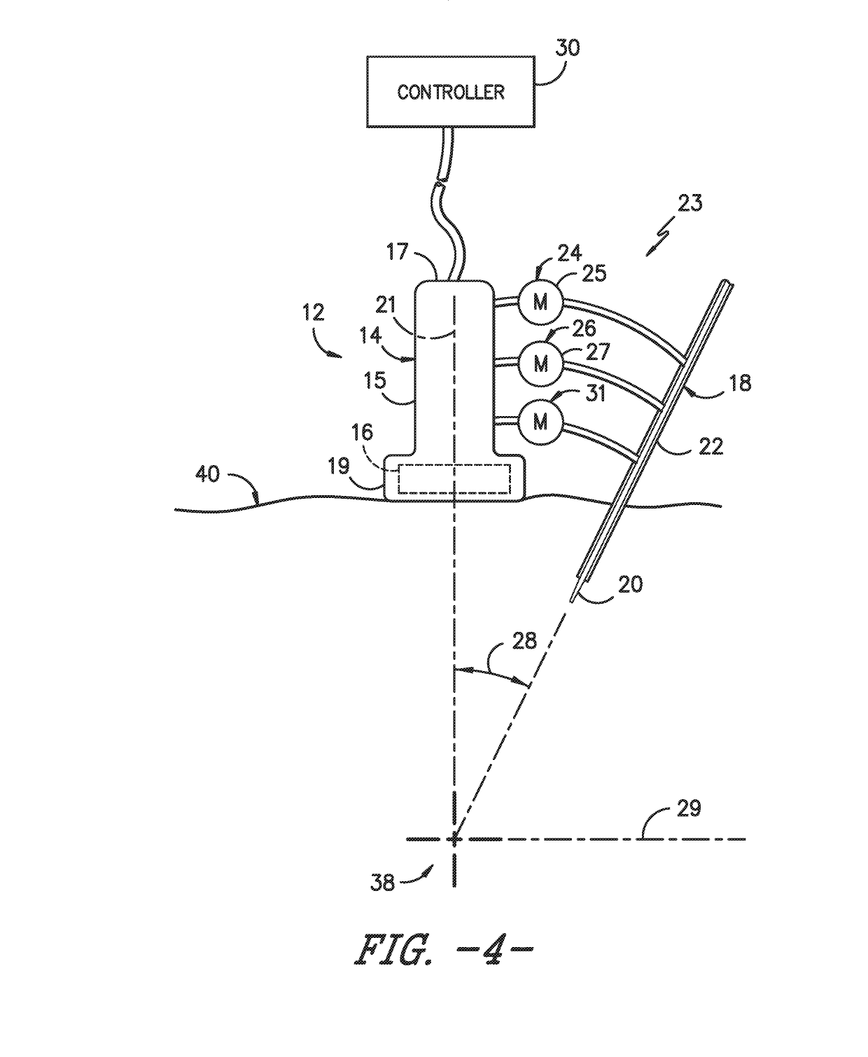

Referring particularly to FIGS. 1 and 3, the ultrasound imaging system 10 may also include a needle guide assembly 18 communicatively coupled to the ultrasound probe 12, at least one actuator component 23 configured with the needle guide assembly 18, and a controller 30. Thus, the controller 30 is configured to determine an insertion angle 28 and a lateral position 29 for the needle guide assembly 18 with respect to the ultrasound probe 12 and the target site 38. Further, the controller 30 is configured to control the actuator component 23 based on the insertion angle 28 and/or the lateral position 29 so as to locate the needle guide assembly 18 at the target site 38 during a medical procedure.

More specifically, as shown, the needle guide assembly 18 may include, at least, a needle 20 and a catheter 22. As such, it should be understood that the needle 20 as well as the catheter 22 of the needle guide assembly 18 can be inserted into the patient in any particular order or simultaneously. For example, in one embodiment, the ultrasound imaging system 10 may include an over-the-needle (OTN) catheter assembly in which the catheter 22 is coaxially mounted over the needle 20. Alternatively, the needle 20 may be mounted over the catheter 22. In such embodiments, the needle 20 may act as an introducer such that it places the catheter 22 at the target site 38 and is later removed.

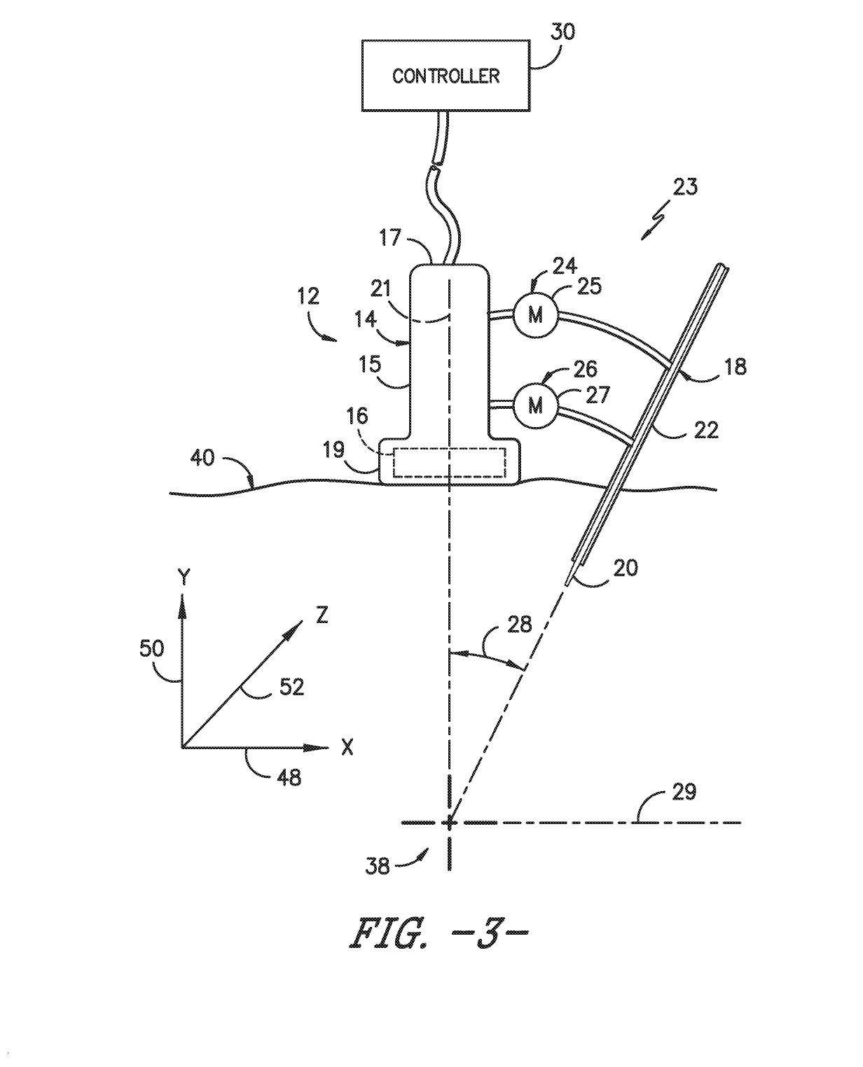

Referring now to FIG. 2, there is illustrated a block diagram of one embodiment of suitable components that may be included within the controller 30 in accordance with aspects of the present subject matter. As shown, the controller 30 may include one or more processor(s) 32 and associated memory device(s) 33 configured to perform a variety of computer-implemented functions (e.g., performing the methods, steps, and the like and storing relevant data as disclosed herein). Additionally, the controller 30 may also include a communications module 34 to facilitate communications between the controller 30 and the various components of the system 10. Further, the communications module 34 may include a sensor interface 35 (e.g., one or more analog-to-digital converters) to permit signals transmitted from the probe 12 to be converted into signals that can be understood and processed by the processor(s) 32. In addition, as shown, the ultrasound imaging system 10 may also include a user interface 36 (FIG. 1) configured to display an image of the target site 38 to a user during the medical procedure (or before or after). More specifically, in certain embodiments, the user interface 36 may be configured to allow a user to manipulate the 3D image according to one or more user preferences.

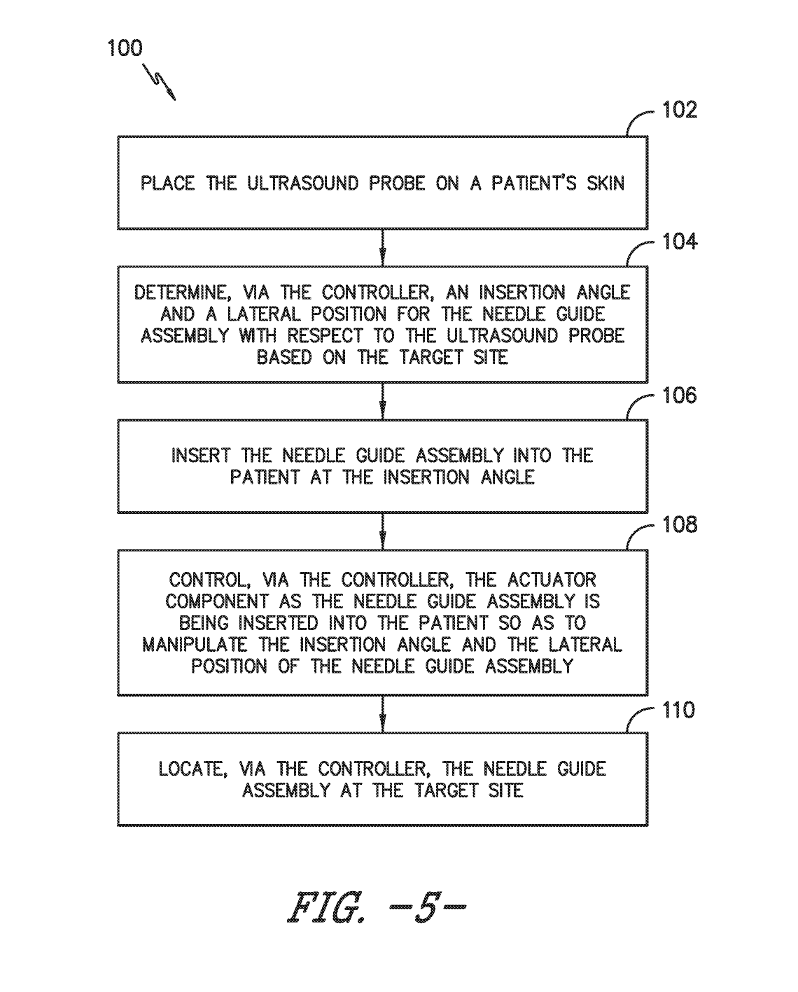

Referring particularly to FIGS. 3 and 4, the actuator component 23 may include, at least, a first actuator device 24 and a second actuator device 26. More specifically, the first actuator device 24 is configured to move the needle guide assembly 18 with respect to a first axis 48 of the ultrasound probe 12, whereas the second actuator device 26 is configured to move the needle guide assembly 18 with respect to a second axis 50 of the ultrasound probe 12. More specifically, in certain embodiments, the first and second actuator devices 24, 26 may include a first motor 25 configured to control the insertion angle and a second motor 27 configured to control the lateral position, respectively. The motors described herein may include any suitable type of motor including but not limited to electric motors (i.e. a DC or AC motor), hydraulic motors, pneumatic motors, or any other suitable motor. In such embodiments, the controller 30 may be further configured to control the first and second actuator devices 24, 26 so as to maintain the needle guide assembly 18 in-plane with respect to the first and second axes 48, 50 of the ultrasound probe 12.

In further embodiments, the ultrasound imaging system 10 may also include an optional third actuator device 31 or motor for manipulating the needle guide assembly 18 with respect to a third axis 52 of the ultrasound probe 12. In such embodiments, the first, second, and third actuator devices 24, 26, 31 are configured to move the needle guide assembly 18 out-of-plane with respect to the ultrasound probe 12.

In additional embodiments, the controller 30 is further configured to track the needle guide assembly 18 as the assembly 18 is being inserted into the patient. As such, in certain embodiments, the controller 30 is further configured to adjust the insertion angle and/or the lateral position of the needle guide assembly 18 as the assembly 18 is being inserted into the patient during the medical procedure as needed. Further, in particular embodiments, the controller 30 is further configured to determine the insertion angle as a function of a depth of the target site 38 within the patient.

Referring now to FIG. 5, a flow diagram of one embodiment of a method 100 for locating a needle guide assembly 18 of an ultrasound imaging system 10 at a target site 38 of a patient during an ultrasound-guided medical procedure is illustrated. As described herein, the ultrasound imaging system 10 also includes an ultrasound probe 12, an actuator component 23 configured with the needle guide assembly 18, and a controller 30. Thus, as shown at 102, the method 100 includes placing the ultrasound probe 12 on a patient's skin 40 (FIGS. 3 and 4). Further, as shown at 104, the method 100 includes determining, via the controller 30, an insertion angle 28 and a lateral position 29 for the needle guide assembly 18 with respect to the ultrasound probe 12 based on the target site 38. As shown at 106, the method 100 includes inserting the needle guide assembly 18 into the patient at the insertion angle 28. As shown at 108, the method 100 includes controlling, via the controller 30, the actuator component 23 as the needle guide assembly 18 is being inserted into the patient so as to manipulate the insertion angle 28 and the lateral position 29 of the needle guide assembly 18 as needed. As shown at 110, the method 100 includes locating, via the controller 30, the needle guide assembly 18 at the target site 38.

In addition, in one embodiment, the method 100 may include scanning, via the ultrasound probe 12, images of the target site 38 and generating, via the controller 30, an ultrasound image based on the images during the ultrasound-guided medical procedure.

In further embodiments, the method 100 may include controlling, via the controller 30, first and second actuator devices 24, 26 so as to maintain the needle guide assembly 18 in-plane with respect to the first and second axes 48, 50 of the ultrasound probe 12. In addition, in particular embodiments, the method 100 may further include manipulating, via a third actuator device 31 or motor, the needle guide assembly 18 with respect to a third axis 52 of the ultrasound probe 12.

In additional embodiments, the method 100 may also include adjusting, via the controller 30, the insertion angle or the lateral position of the needle guide assembly 18 based on the target site 38 as the needle guide assembly 18 is being inserted into the patient during the medical procedure. In yet another embodiment, the method 100 may include determining, via the controller 30, the insertion angle 28 of the needle guide assembly 18 as a function of a depth of the target site 38 within the patient.

In still a further embodiment, the method 100 may include tracking, via the controller 30, the needle guide assembly 18 as the assembly 18 is being inserted into the patient. In another embodiment, the method 100 may also include generating, via a user interface 36, a display of the target site 38 for a user.

While various patents have been incorporated herein by reference, to the extent there is any inconsistency between incorporated material and that of the written specification, the written specification shall control. In addition, while the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

1. An ultrasound imaging system, comprising:
an ultrasound probe comprising:
a transducer housing comprising a body extending from a proximal end to a distal end along a longitudinal axis, the distal end comprising an internal cavity, and
a transducer transmitter configured within the cavity, the transducer transmitter configured to scan a target site of a patient;
a needle guide assembly communicatively coupled to the ultrasound probe;
at least one actuator component configured with the needle guide assembly; and,
a controller configured to determine an insertion angle and a lateral position for the needle guide assembly with respect to the ultrasound probe based on the target site and control the actuator component based on the insertion angle and the lateral position so as to locate the needle guide assembly at the target site during a medical procedure.

2. The ultrasound imaging system of claim 1, wherein the needle guide assembly comprises, at least, a needle guide and a catheter.

3. The ultrasound imaging system of claim 1, wherein the actuator component comprises a first actuator device configured to move the needle guide assembly with respect to a first axis of the ultrasound probe and a second actuator device configured to move the needle guide assembly with respect to a second axis of the ultrasound probe.

4. The ultrasound imaging system of claim 3, wherein the controller is further configured to control the first and second actuator devices so as to maintain the needle guide assembly in-plane with respect to the first and second axes of the ultrasound probe.

5. The ultrasound imaging system of claim 3, wherein the first and second actuator devices comprise a first motor configured to control the insertion angle and a second motor configured to control the lateral position, respectively.

6. The ultrasound imaging system of claim 5, further comprising a third actuator device configured to move the needle guide assembly with respect to a third axis of the ultrasound probe.

7. The ultrasound imaging system of claim 2, wherein the controller is further configured to adjust at least one of the insertion angle or the lateral position as a function of the target site as the needle guide assembly is being inserted into the patient during the medical procedure.

8. The ultrasound imaging system of claim 7, wherein the controller is further configured to determine the insertion angle as a function of a depth of the target site within the patient.

9. The ultrasound imaging system of claim 2, wherein the ultrasound probe is further configured to track the needle guide assembly as the needle guide assembly is being inserted into the patient.

10. The ultrasound imaging system of claim 1, further comprising a user interface configured to display the target site to a user during the medical procedure.

11. The ultrasound imaging system of claim 1, wherein the medical procedure comprises a peripheral nerve block procedure.

12. A method for locating a needle guide assembly of an ultrasound imaging system at a target site of a patient during an ultrasound-guided medical procedure, the ultrasound imaging system also having an ultrasound probe, an actuator component configured with the needle guide assembly, and a controller, the method comprising:
placing the ultrasound probe on a patient's skin;
determining, via the controller, an insertion angle and a lateral position for the needle guide assembly with respect to the ultrasound probe based on the target site;
inserting the needle guide assembly into the patient at the insertion angle;
controlling, via the controller, the actuator component as the needle guide assembly is being inserted into the patient so as to manipulate the insertion angle and the lateral position of the needle guide assembly; and
locating, via the controller, the needle guide assembly at the target site.

13. (canceled)

14. The method of claim 12, further comprising scanning, via the ultrasound probe, images of the target site and generating, via the controller, an ultrasound image based on the images during the ultrasound-guided medical procedure.

15. The method of claim 14, wherein the actuator component comprises a first actuator device configured to move the needle guide assembly with respect to a first axis of the ultrasound probe and a second actuator device configured to move the needle guide assembly with respect to a second axis of the ultrasound probe.

16. The method of claim 15, further comprising controlling, via the controller, the first and second actuator devices so as to maintain the needle guide assembly in-plane with respect to the first and second axes of the ultrasound probe.

17. The method of claim 15, wherein the first and second actuator devices comprise a first motor configured to control the insertion angle and a second motor configured to control the lateral position, respectively.

18. The method of claim 17, further comprising manipulating, via a third actuator device, the needle guide assembly with respect to a third axis of the ultrasound probe.

19. The method of claim 15, further comprising adjusting, via the controller, at least one of the insertion angle or the lateral position as a function of the target site as the needle guide assembly is being inserted into the patient during the medical procedure.

20. The method of claim 19, further comprising determining, via the controller, the insertion angle as a function of a depth of the target site within the patient.

21. The method of claim 14, further comprising tracking, via the ultrasound probe, the needle guide assembly as the needle guide assembly is being inserted into the patient.

22-32. (canceled)
